(19) United States
(12) Patent Application Publication
(10) Pub. No.: US 2007/0163573 A1
(43) Pub. Date: Jul. 19, 2007

(54) WOUND CLEANING AND DECONTAMINATION DEVICE AND METHOD OF USE THEREOF

(75) Inventors: Michael D. RAINONE, Palestine, TX (US); Brook A. GRISHAM, Chandler, TX (US)

Correspondence Address: HAHN LOESER & PARKS, LLP, One GOJO Plaza, Suite 300, AKRON, OH, 44311-1076, US

(73) Assignee: ACT SEED TECHNOLOGY FUND LLC, Palestine, TX (US)

(21) Appl. No.: 11/623,969

(22) Filed: Jan. 17, 2007

Related U.S. Application Data
(60) Provisional application No. 60/759,702, filed on Jan. 18, 2006.

(51) Int. Cl. A61M 11/00

(52) U.S. Cl. 128/200.14

(57) ABSTRACT

A wound cleaning and decontamination device and method is provided using directional control provided by a fluid oscillator providing a pulsating stream of droplets capable of inducing a vibrating or trampolining effect on the surface being cleaned. The device may include a pulsital oscillating nozzle supported by a portable housing, and a reservoir of cleaning fluid. The pulsating stream oscillates at a resonant frequency of the surface being cleaned, which may be between 20 and 80 Hz. The method may include using a pulsital oscillating nozzle for driving a disinfecting solution into the interstices of an epidermis layer of skin.

This U.S. patent application claims the benefit of U.S. Provisional Patent Application Ser. No. 60/759,702, which was filed on Jan. 18, 2006, and incorporated herein by reference.

### BACKGROUND AND SUMMARY OF THE DISCLOSURE

The present invention relates to devices and methods for washing and decontaminating, and more particularly to devices and methods for debridement and decontamination of skin by irrigation.

Cleaning of skin surfaces is a regular function in medical and other activities to reduce contamination and infection. Infectious bacteria and viruses and other pathogens may be transmitted by contact with blood, saliva, and other bodily fluids. Proper washing and decontaminating of skin reduces infections and promotes healing.

Wound debridement involves cleaning a wound and removing damaged or contaminated tissue. Wounds, such as lacerations, burns, infected tissue, bedsores, and other wounds, may be cleaned and decontaminated using treatments such as mechanical and surgical debridement, and biological and chemical agents. Cleaning devices and methods may be used in clinical settings such as in hospitals, clinics, and doctor's offices, in home healthcare, and in other settings.

Skin is made up of layers, where the top epidermal layers include layers of mostly dead cells containing the protein keratin. The lower epidermal layers include a basal cell layer of living cells that regenerate the skin. The upper layer of mostly dead cells, such as the stratum corneum, can harbor various bacteria and flora within the interstices between cells. Skin surfaces and interstices may have potentially infectious pathogens such as Staphylococcus aureus, or “staph,” Streptococcus pyogenes, or “strep,” Escherichia coli, or “e. coli,” and many others. Some current methods of cleaning and debridement may clean the outer surface of the skin, but fail to remove various bacteria from within the layers and interstices of skin cells.

Current methods and products for cleaning and debridement include a simple rinsing approach with cumbersome processes. Conventional medical strategies for cleaning and decontaminating skin surfaces, such as by scrubbing, require diligence over an extended period, such as 5 to 15 minutes. The effectiveness of the decontamination may not be satisfactory when performed by less diligent or less experienced personnel. Some products and methods debride or clean randomly and non-selectively, possibly injuring or removing new tissue along with necrotic tissue.

Pressurized and pulsating jets of fluids have been used for cleaning and irrigating. While the pulsating jets of fluid appear to be satisfactory for some applications, when used for washing skin the pulsating fluid repeatedly impacting a surface has been found to deleteriously force bacteria and debris into the pores and interstices of the skin layers instead of washing it away. Further, personnel using such devices may have difficulty discerning the required amount, flow rate and delivery accuracy for effectively applying fluids, and difficulty discerning the degree of effectiveness in the resulting debridement or decontamination.

U.S. Pat. Nos. 6,110,292 and 6,176,941, both to Jewett, et al., disclose a device for washing hands and forearms. The Jewett, et al., device uses multiple nozzles fixed in a chamber, each nozzle being set at an angle to the surface being cleaned. U.S. Pat. Nos. 6,110,292 and 6,176,941 teach a brushless cleaning device and methods of use thereof requiring a source of water such as municipal water, and dispensing of a sequence of three different fluids to achieve cleaning. There is no teaching or suggestion of a self contained or portable device.

Fluidic oscillators have been disclosed in U.S. Pat. Nos. Re. 33,159 and Re. 33,448, both to Bauer. The fluidic oscillator, or pulsital oscillating nozzle as further described below, emitting a pulsating stream of droplets creates a resonant effect and a subsequent vibration or trampolining of skin tissue or other surfaces. While the skin is vibrating or trampolining, disinfecting fluids can be pushed into the interstices between skin cells and layers.

Further, spraying a stream of fluid from the pulsital oscillating nozzle over skin having certain wounds may aid in healing. When an open wound is in the skin, a body reacts by initiating a coagulation process, where fibrins are deposited over exposed subsurface tissue. The fibrins coagulate blood in the exposed tissue to initiate healing. However, when two severed tissues are sutured together, the diminished blood flow caused by fibrin can slow healing between the severed surfaces. We have found that when the pulsating stream from the pulsital oscillating nozzle cleanses certain coagulating wounds and exposed subsurface tissue, the pulsating stream debrides fibrins from the wound surfaces and stimulates the tissue. Such stimulated tissue may be sutured together resulting in improved blood flow between the wound surfaces and faster healing.

In view of the foregoing, the present disclosure is directed to a wound cleaning and decontamination system incorporating a trampolining effect on tissue and other surfaces. A particular embodiment of a self contained wound cleaning and decontamination device may comprise:
a. a reservoir comprising a predetermined amount of a cleaning fluid at a selected pressure;
b. a pulsital oscillating nozzle operatively connected to the reservoir, the nozzle being capable of directing the cleaning fluid in a pulsating oscillating stream having a frequency about the resonant frequency of a surface being cleaned;
c. a flow control mechanism operatively controlling the flow to the nozzle; and
d. a portable housing supporting the nozzle.

A particular embodiment of a method of decontaminating pliant skin surfaces may comprise the steps of:
a. selecting a target skin surface having an epidermis layer comprising interstices beneath the skin surface;
b. providing a decontamination device comprising:
   a reservoir comprising a predetermined amount of a cleaning fluid;
   a pulsital oscillating nozzle operatively connected to the reservoir, the nozzle being capable of directing the cleaning fluid in a pulsating oscillating stream having a frequency about a resonant frequency of the target skin surface; and a portable housing supporting the nozzle;
c. applying a disinfecting solution to the target skin surface;
d. spraying the cleaning fluid over the target skin surface for a predetermined period from the pulsital oscillating nozzle and causing the target skin surface to vibrate at a resonant frequency; and
e. driving the disinfecting solution into the interstices of the epidermis layer.

In another embodiment, a method of decontaminating pliant skin surfaces may comprise the steps of:
a. selecting a target skin surface having a coagulating wound;
b. providing a decontamination device comprising:
   a reservoir comprising a predetermined amount of a cleaning fluid;
   a pulsital oscillating nozzle operatively connected to the reservoir, the nozzle being capable of directing the cleaning fluid in a pulsating oscillating stream having a frequency between about 20 and 80 Hz; and a portable housing supporting the nozzle;
c. applying a disinfecting solution to at least a portion of the target skin surface;
d. spraying the cleaning fluid over the target skin surface for a predetermined period in an oscillating stream having a frequency between about 20 and 80 Hz from the pulsital oscillating nozzle; and
e. stimulating and removing fibrin from the coagulating wound.

### BRIEF DESCRIPTION OF THE DRAWINGS

The invention is described in more detail with reference to the accompanying drawings, in which:

FIG. 1 is a partial sectional view showing major components of a wound cleaning and decontamination device;

FIG. 2 is a section through a pulsital oscillating nozzle;

FIG. 3 is a section through a second embodiment of a pulsital oscillating nozzle;

FIG. 4 is a partial cross sectional view through a second embodiment of the present disclosure;

FIG. 5 is a partial cross sectional view through an embodiment of the present disclosure using a pressurized gas to pressurize the device; and

FIG. 6 is a perspective view of the device of FIG. 1 with a slash guard attached.

### DETAILED DESCRIPTION OF THE DRAWINGS

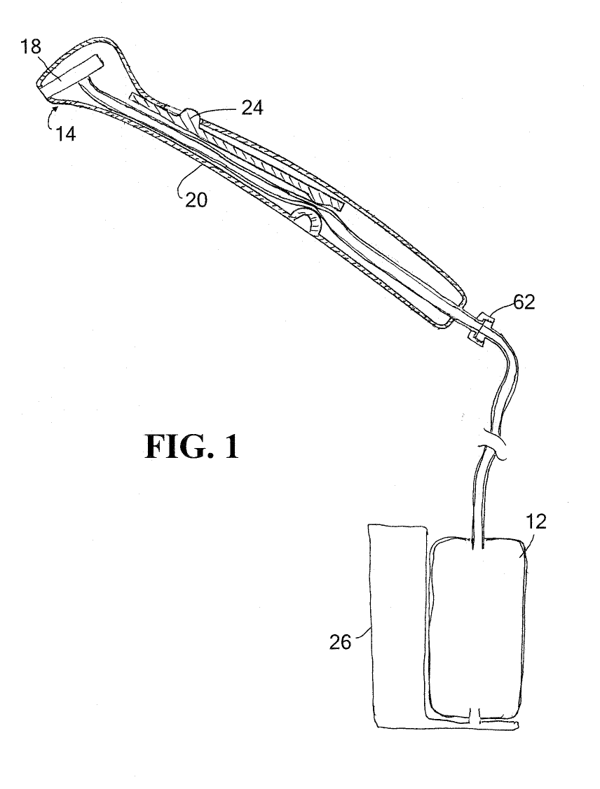

A wound cleaning and decontamination device 10 shown in FIG. 1 includes a reservoir 12 containing a cleaning fluid in communication with a nozzle assembly 14 comprising at least one fluid oscillator, or pulsital oscillating nozzle 18. The decontamination device 10 may have a handling grip assembly, or housing assembly 20. The housing assembly 20 may be a portable housing assembly 20 with features for manually controlling the wound cleaning and decontamination device 10. Alternately, the housing assembly 20 may include features for control via an automatic, remote manipulation approach.

Measurement and inducement of fluid flow may be accomplished with an operating control assembly. The operating control assembly may include at least one flow control mechanism 24, a pressurizing device 26, and the nozzle assembly 14 to control the pressure, flow and flow rate of a fluid emitted from the device. The operating control assembly may be manually controllable or preset, and designed to translate positions of a device, such as the flow control mechanism 24 or a portion thereof, into a fluid displacement flow to the nozzle assembly 14. The flow control mechanism 24 may be used for allowing transfer of one or more fluids to the nozzle assembly 14, where the fluid exits.

The flow control mechanism 24 may be a valve operatively controlling the flow from the reservoir 12 to the nozzle assembly 14. In the embodiment of FIG. 1, the flow control mechanism 24 includes a sliding component that pinches a conduit to stop flow through the conduit. The flow control mechanism 24 may be any suitable valve capable of controlling the flow of fluid to the nozzle assembly 14.

The wound cleaning and decontamination device 10 delivers fluid from the reservoir 12 to the pulsital oscillating nozzle 18 under pressure. The wound cleaning and decontamination device 10 may include the pressurizing device 26 capable of pressurizing the reservoir 12 to a predetermined pressure corresponding to a desired spray frequency from the pulsital oscillating nozzle 18. The pressurizing device may be capable of pressurizing the reservoir to between about 30 to 70 pounds per square inch. Optionally, the reservoir 12 may be a pre-pressurized container.

As shown in FIG. 5, the pressurizing device 26′ may be a container of pressurized gas in communication with the reservoir 12, such as a canister of carbon dioxide or compressed air capable of pressurizing the reservoir 12. Alternately, the pressurizing device 26 may be a pump.

The operating control assembly may provide operator control by way of buttons, touch pads, sliding components, switches, or other mechanical or electronic controls. The operating control assembly may further provide feedback to the operator, such as by audible constant or variable tones, or a digital display. Power sources and associated electronics may be provided, such as but not limited to replaceable or rechargeable batteries or direct current power connections.

Optionally, the operating control assembly may include sensors for monitoring and reporting operational status, including but not limited to amount of fluid in the reservoir 12 or amount of fluid sprayed, amount of time elapsed, or other status.

In some embodiments, there may be multiple operating control assemblies incorporated into the wound cleaning and decontamination device 10 independently or in combination providing a stream of fluid. Multiple operating control assemblies may be used to deliver multiple fluid flows for applications such as applying fluids onto non-constant surfaces having more than one resonant frequency. Multiple pulsital oscillating nozzles may provide fluid flow in one or more streams having variable or fixed and intermixed, interacting, or separate spray patterns.

The nozzle assembly 14 may have a configuration of one or more pulsital oscillating nozzles 18 to apply a fluid with combined, independent, or complementary fluid streams and patterns to a target surface of human or other organic tissue, or to inorganic surfaces. The pulsital oscillating nozzles 18 emit one or more streams of droplets, or pulsating streams, that create a resonant effect and a subsequent vibration or trampolining of the pliant surface being sprayed.

The directional control of the fluid spray stream provided by the nozzle assembly 14, which includes the pulsital oscillating nozzle 18, for application and delivery of the fluid delivers one or more sprays emitting flow streams in a fan pattern or other 2 or 3 dimensional patterns.

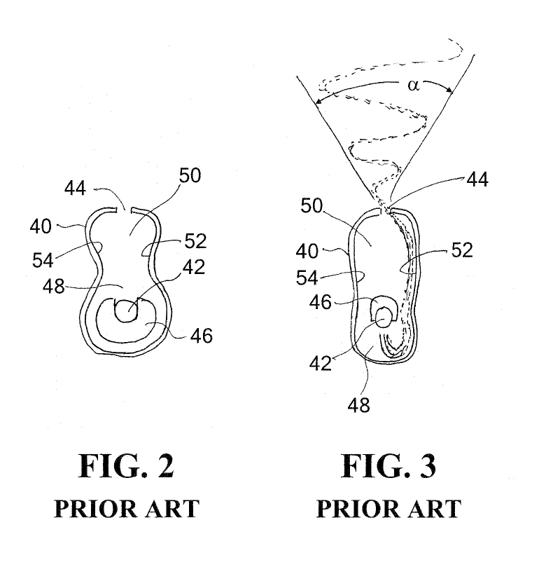

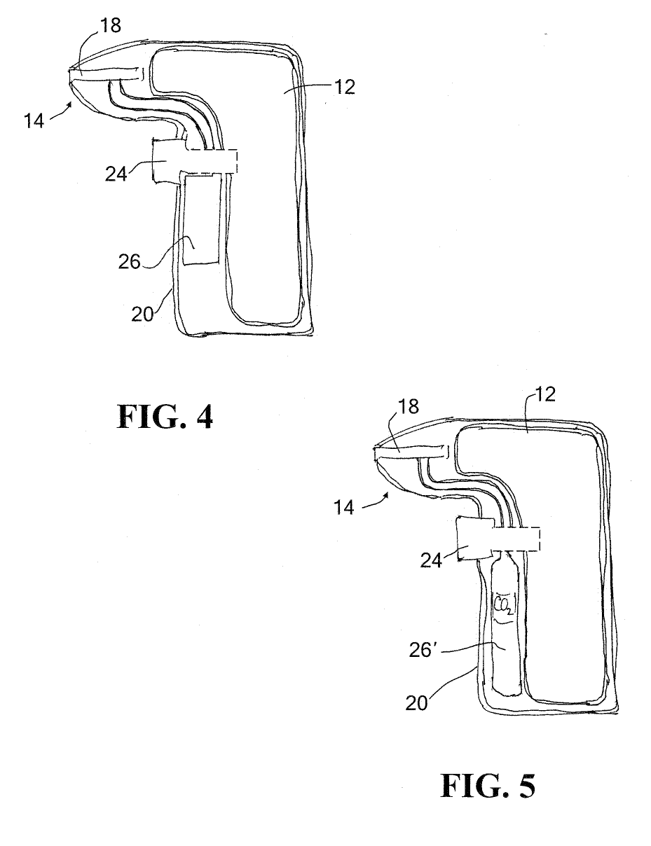

The pulsital oscillating nozzle 18, such as the prior art embodiments shown in FIGS. 2 and 3, may have a generally hollow nozzle body 40 having an inlet 42 and an outlet 44. The flow of pressurized fluid through inlet 42 may be directed by a diverter 46 into an entry cavity region 48 in the nozzle body 40. The entry cavity region 48 may be located in a direction toward the outlet 44 as shown in FIG. 2. Alternately, the entry cavity region 48 may be in a direction away from the outlet 44 as shown in FIG. 3. From the entry cavity region 48, the flow is directed to an exit cavity region 50 in the direction of the outlet 44. The flow of fluid in the nozzle body 40 is not stable, and vortices disrupt the flow, causing the flow of fluid to momentarily travel along one side 52 of the exit cavity region 50, and then to momentarily travel along an opposite side 54 of the exit cavity region 50. The flow alternates from the side 52 to the opposite side 54 rapidly as the flow exits the outlet 44. The alternating flow inside the nozzle body 40 causes the flow to exit the outlet 44 in a stream that oscillates back and forth, or laterally oscillates. Further, as the flow inside the nozzle body 40 moves from one side of the exit cavity region 50 to the other, the flow through the outlet 44 may instantaneously diminish, resulting in the stream being a pulsating stream made up of pulses or droplets of fluid.

The shape and size of the entry cavity region 48, the diverter 46, the exit cavity region 50 and the outlet 44 may be varied to produce different flow patterns and oscillation frequencies. By directing the flow from the inlet 42 in the direction away from the outlet 44 as shown in FIG. 3, the distance that the fluid flows within the nozzle body is increased. A longer flow length in the nozzle body may provide a lower oscillation frequency.

The pulsating stream of fluid from the pulsital oscillating nozzle 18 is made up a series of droplets that exit the outlet 44 one after another. The oscillating flow of fluid causes the droplets to exit the outlet 44 such that each exiting droplet may exit at a slightly different trajectory angle as the stream of droplets oscillates. The pulsital oscillating nozzle 18 may be configured such that the droplets have a sufficient mass and velocity to induce vibration or a trampolining of the surface being cleaned.

The laterally oscillating stream of fluid may exit the outlet 44 as a fan pattern, having a width defined by an angle α, as shown in FIG. 3. Various two and three dimensional spray patterns may be achieved by varying the dimensions of the pulsital oscillating nozzle 18.

Pressurized fluid enters the inlet 42. The frequency of the stream of fluid from the outlet 44 may be varied by varying the inlet pressure of the fluid. The pressure at inlet 42 may be between 30 and 70 pounds per square inch, and the frequency of the fluid stream at the outlet 44 may be between about 20 and 80 Hz, or about the resonant frequency of the surface being cleaned.

The portable housing assembly 20 may have a shape capable of being a hand-held housing. The housing assembly 20 may include a gripping handle 22 having a formed material suitable for holding or gripping by an operator's hand. The portable or hand-held housing assembly 20 may have a shape that allows gripping of the device without a gripping handle 22. In some embodiments, the housing assembly 20 may include an attachment assembly (not shown) for mounting the device 10 in an automated, remotely operated, wound cleaning and decontamination application.

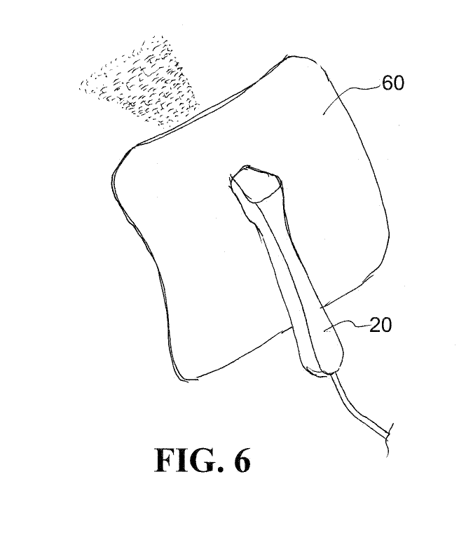

In the embodiment of FIG. 6, the wound cleaning and decontamination device 10 may comprise one or more shields, protective coverings, or splash guards, collectively referred to herein as splash guards 60. Splash guards 60 may be capable of deflecting various fluids and exudates splashing from the surface being cleaned. The splash guards 60 may at least partially cover various portions and elements of the device 10. The splash guards 60 may include one or more components at least partially surrounding the nozzle and being affixed to the housing assembly 20. The splash guard 60 may have components that attach to each other. In some embodiments, the splash guard 60 and other protective components may be omitted.

The splash guard 60 may have a shape about 8 to 10 inches across, and at least partially surrounding the nozzle and being affixed to the housing assembly 20. The housing assembly 20 and splash guards 60 may be made from a thermoplastic or other suitable material. The housing assembly 20 and splash guards 60 may be made from an impact and chemical resistant material.

The wound cleaning and decontamination device 10 may be a self-contained, disposable system. The reservoir 12 may be a single use fluid reservoir cartridge, where the fluid may be contained until use. In a single use embodiment, the reservoir 12 may be pre-pressurized. Alternately, the reservoir 12 may be replaceable or rechargeable. Replaceable or reusable connectors may be used for removably connecting the reservoir 12 to the operating control assembly.

The reservoir 12 may be partially or completely enclosed within the housing assembly 20. Alternately, the reservoir 12 may be distal from the housing assembly 20, and connected to the nozzle assembly 14 by a fluid conduit or tube. Replaceable or reusable connectors 62 may be used for removably connecting the fluid conduit or tube to the reservoir 12 and the nozzle assembly 14.

The operating control assembly may include an electronic wired or wireless communication module that would enable two-way communication of information and control between the wound cleaning and decontamination device 10 and a remote control interface unit. The remote control interface may actuate the wound cleaning and decontamination device 10 without direct human contact or intervention during the application of fluids. An electronic wired or wireless communication function may enable communication of data and control information to a remote data acquisition system.

The wound cleaning and decontamination device 10 may be used to induce a vibrating or trampoline effect on skin or exposed subsurface tissues or other surfaces resulting in removal of debris and other undesirable elements and/or contaminants. Single or multiple pulsital oscillating nozzles 18 may be used to deliver one or more fluids to a target surface by emitting a stream of droplets that creates a resonant effect and a subsequent vibration or trampolining of differing types of tissue or other surfaces. When cleaning fluid is applied to a target surface, impact forces applied from the stream of droplets is continuously moving as the stream of droplets oscillates back and forth, and the flow of cleaning fluid removes material from that surface or in proximity above or below that surface.

When decontaminating a skin surface, the skin surface is sprayed with a pulsating and oscillating stream from the pulsital oscillating nozzle 18. The pulsating and oscillating spray applied to the skin surface may cause the skin to vibrate at a resonant frequency. To make a skin surface vibrate at the resonate frequency, the pulsating stream leaving the outlet 44 may have frequency about the resonant frequency of the surface being cleaned. The resonating frequency for some skin surfaces may be about 58 Hz. Skin surfaces may resonate in a range between about 20 and 80 Hz.

In one method of decontaminating a pliant skin surface, a disinfecting solution may be applied to a target skin surface. Then, the pulsital oscillating nozzle 18 directs a cleaning fluid in an oscillating stream having a frequency between about 20 and 80 Hz over the target skin surface for a predetermined period, causing the target skin surface to vibrate at a resonant frequency. The cleaning fluid applied to the vibrating skin surface drives the disinfecting solution into the interstices of the epidermis layer. In one method, the predetermined period is about 15 seconds. The predetermined period may be up to about 60 seconds, 90 seconds, or more.

The disinfecting solution may include a disinfectant such as, but not limited to iodine, povidone iodine, chlorhexidine gluconate, and 70% isopropyl alcohol. The disinfecting solution may be swabbed onto the skin surface using a surgical scrub. Alternately, the disinfecting solution may be in the reservoir 12 and then sprayed onto the skin surface using the wound cleaning and decontamination device 10.

After the disinfecting solution is applied to the target skin surface, the disinfecting solution may be driven into the interstices of the skin by spraying a cleaning fluid such as sterile water over the skin surface in a pulsating stream having a frequency about the resonant frequency of the surface being cleaned, such as in a range between about 20 and 80 Hz. Alternately, the stream of fluid may be a detergent or other cleaning fluid.

In one method of decontaminating pliant skin surfaces having a coagulating wound, the disinfecting solution is applied to at least a portion of the target skin surface. Then, the pulsital oscillating nozzle 18 directs a cleaning fluid in an oscillating stream having a frequency between about 20 and 80 Hz over the target skin surface and coagulating wound for a predetermined period, stimulating the wound and removing fibrin from the coagulating wound.

The methods of decontaminating pliant skin surfaces may be performed using the portable or hand-held wound cleaning and decontamination device, whereby the operator's hand motion determines the angle and sweep rate of the spray of cleaning fluid over the target surface.

The present disclosure provides wound cleaning and decontamination devices and methods for efficiently and effectively cleaning and decontaminating skin surfaces and debriding wounds, using directional control provided by a fluid oscillator for providing a pulsating stream of fluid capable of inducing a vibrating or trampoline effect on the skin or exposed tissue or other surface for removing debris, undesirable elements, and contaminants.

While the invention has been described with detailed reference to one or more embodiments, the disclosure is to be considered as illustrative and not restrictive. Modifications and alterations will occur to those skilled in the art upon a reading and understanding of this specification. It is intended to include all such modifications and alterations in so far as they come within the scope of the claims, or the equivalence thereof.

What is claimed is:

1. A method of decontaminating pliant skin surfaces comprising the steps of:
a. selecting a target skin surface having an epidermis layer comprising interstices beneath the skin surface;
b. providing a decontamination device comprising:
i. a reservoir comprising a predetermined amount of a cleaning fluid;
ii. a pulsital oscillating nozzle operatively connected to the reservoir, the nozzle being capable of directing the cleaning fluid in a pulsating oscillating stream having a frequency about a resonant frequency of the target skin surface; and
iii. a portable housing supporting the nozzle;
c. applying a disinfecting solution to the target skin surface;
d. spraying the cleaning fluid over the target skin surface for a predetermined period from the pulsital oscillating nozzle and causing the target skin surface to vibrate at a resonant frequency; and
e. driving the disinfecting solution into the interstices of the epidermis layer.

2. The method according to claim 1, the step of spraying the cleaning fluid further comprising the step of:
spraying the cleaning fluid over the target surface at an angle and a sweep rate determined by an operator's hand motion.

3. The method according to claim 1, the frequency being between about 20 and 80 Hz.

4. The method according to claim 1, the predetermined period being about 15 seconds.

5. The method according to claim 1, the cleaning fluid comprising sterile water.

6. The method according to claim 1, the step of applying a disinfecting solution to the target area comprising the step of:
spraying the disinfecting solution over the target surface for a predetermined period from a second pulsital oscillating nozzle capable of directing the disinfecting solution in a pulsating oscillating stream having a frequency between about 20 and 80 Hz.

7. A method of decontaminating pliant skin surfaces comprising the steps of:
a. selecting a target skin surface having a coagulating wound;
b. providing a decontamination device comprising:
i. a reservoir comprising a predetermined amount of a cleaning fluid;
ii. a pulsital oscillating nozzle operatively connected to the reservoir, the nozzle being capable of directing the cleaning fluid in a pulsating oscillating stream having a frequency between about 20 and 80 Hz; and
iii. a portable housing supporting the nozzle;
c. applying a disinfecting solution to at least a portion of the target skin surface;
d. spraying the cleaning fluid over the target skin surface for a predetermined period in an oscillating stream having a frequency between about 20 and 80 Hz from the pulsital oscillating nozzle; and
e. stimulating and removing fibrin from the coagulating wound.

8. The method according to claim 7, the cleaning fluid comprising sterile water.

9. The method according to claim 7, the predetermined period being about 15 seconds.

10. A self contained decontamination device comprising:
a. a reservoir comprising a predetermined amount of a cleaning fluid at a selected pressure;
b. a pulsital oscillating nozzle operatively connected to the reservoir, the nozzle being capable of directing the cleaning fluid in a pulsating oscillating stream having a frequency about the resonant frequency of a surface being cleaned;
c. a flow control mechanism operatively controlling the flow to the nozzle; and
d. a portable housing supporting the nozzle.

11. The device according to claim 10, further comprising:
a pressurizing device connected to the reservoir capable of pressurizing the reservoir to a pressure of about 30 to 70 pounds per square inch.

12. The device according to claim 11, the pressurizing device being a container of pressurized gas.

13. The device according to claim 11, the pressurizing device being a pump.

14. The device according to claim 10, further comprising:
a splash guard at least partially surrounding the nozzle and being affixed to the housing.

15. The device according to claim 10, the frequency being between about 20 to 80 Hz to correspond with a resonant frequency of human skin.

16. The device according to claim 10, the reservoir being at least partially within the housing.

17. The device according to claim 10, the reservoir being distal from the housing and connected to the nozzle by a fluid conduit.

18. The device according to claim 10, the cleaning fluid comprising a disinfectant.

19. The device according to claim 10, the housing comprising a shape providing a gripping handle.
